(19) United States
(12) Patent Application Publication
(10) Pub. No.: US 2026/0066093 A1
(43) Pub. Date: Mar. 5, 2026

(54) SYSTEMS AND METHODS FOR TRANSMISSION OF MEDICAL IMAGE METADATA

(71) Applicant: Stryker Corporation, Portage, MI (US)

(72) Inventors: Marc ANDRÉ, Spiegel b. Bern (CH); Aurelien CHIRON, Santa Clara, CA (US)

(73) Assignee: Stryker Corporation, Portage, MI (US)

(21) Appl. No.: 19/316,329

(22) Filed: Sep. 2, 2025

Related U.S. Application Data
(60) Provisional application No. 63/690,145, filed on Sep. 3, 2024.

(51) Int. Cl. G16H 30/20; G06T 7/00; H04N 7/18

(52) U.S. Cl. CPC G16H 30/20; G06T 7/0012; H04N 7/183; G06T 2207/10016; G06T 2207/10024; G06T 2207/10068; G06T 2207/20081; G06T 2207/30168

(57) ABSTRACT

The present invention generally relates to medical imaging, and more specifically to transmitting and tracking the transmission of medical image data and associated metadata. An exemplary method for transmitting and tracking the transmission of medical image data from a first device to a second device comprises receiving, at the first device, a video frame and frame-specific metadata; in response to receiving the video frame and the frame-specific metadata, generating, at the first device, device identification data of the first device; generating a set of one or more data structures in accordance with a predefined data specification, wherein the set of one or more data structures comprises the frame-specific metadata and the device identification data; and transmitting, by the first device, the set of one or more data structures along with the video frame to the second device.

### CROSS-REFERENCE TO RELATED APPLICATION

This application claims the benefit of U.S. Provisional Application No. 63/690,145, filed Sep. 3, 2024, the entire contents of which is incorporated herein by reference.

### FIELD

The present invention generally relates to medical imaging, and more specifically to transmitting and tracking the transmission of medical image data and associated metadata.

### BACKGROUND

Medical systems, instruments and tools are utilized pre-surgery, during surgery, and post-operatively for various purposes. Some of these medical tools may be used in what are generally termed as endoscopic procedures or open field procedures. For example, endoscopy allows internal features of the body of a patient to be viewed without the use of traditional, fully invasive surgery. Endoscopic imaging systems incorporate endoscopes to enable a surgeon to view a surgical site, and endoscopic tools enable minimally invasive surgery at the site. Such tools may be, for example, shaver-type devices which mechanically cut bone and hard tissue, or radio frequency (RF) probes which are used to remove tissue via ablation or to coagulate tissue to minimize bleeding at the surgical site, for example.

In endoscopic surgery, the endoscope is placed in the body at the location at which it is necessary to perform a surgical procedure. Other surgical instruments, such as the endoscopic tools mentioned above, are also placed in the body at the surgical site. A surgeon views the surgical site through the endoscope to manipulate the tools to perform the desired surgical procedure. Some endoscopes are usable along with a camera head for the purpose of capturing and processing the images received by the endoscope. An endoscopic camera system typically includes a camera head connected to a camera control unit (CCU) by a cable. The CCU processes input image data received from the image sensor of the camera via the cable and then outputs the image data for display. The resolution and frame rates of endoscopic camera systems are ever increasing, and each component of the system must be designed accordingly.

Another type of medical imager that can include a camera head connected to a CCU by a cable is an open-field imager. Open-field imagers can be used to image open surgical fields, for example, for visualizing blood flow in vessels and related tissue perfusion during plastic, microsurgical, reconstructive, and gastrointestinal procedures.

Accordingly, medical image data (e.g., video data) may be generated, transmitted, and/or processed during diagnosis, surgery, and/or post-surgical evaluation. Processing of medical image data allow, for example, real-time and high-precision guidance of a surgeon's instrument during an operation, optical feedback during endoscopy, visualization of fluorescent dye added to contrast anatomical structures, and improvement of surgical operations and protocols. Exemplary imaging processing techniques include automated image sensor alignment, image stabilization, distortion correction, machine-learning-based processing, and fluorescence quantification and normalization. Use of these processing techniques may require that a system accurately and reliably associate each frame of a medical video feed with metadata for that frame.

### SUMMARY

Described herein are devices, systems, and methods for generating and synchronously transmitting frame-specific metadata with medical image data (e.g., intraoperative video frames). An exemplary electronic device can obtain frame-specific metadata and device identification data of the electronic device, and generate one or more data structures (e.g., InfoFrames) in accordance with a predefined data specification. The electronic device can then transmit the one or more data structures along with a video frame to another electronic device.

Various aspects of the present disclosure provide several technical advantages. First, by generating data structures including frame-specific metadata and transmitting the data structures along with video frames, the systems described herein may ensure that video data and associated metadata are received, via transmission of the generated data structures, in a frame-aligned manner without the use of additional hardware. This assurance of temporal alignment of video frames and frame-specific metadata may be important for real-time surgical image processing techniques such as sensor alignment and image stabilization, as well as for post-processing procedures such as the conversion of video data and metadata to a different storage format, machine learning applications, and/or the quantification and normalization of values within a raw fluorescent image frame. Without the ability to send frame-specific metadata with video data on the same communication protocol, alternatives such as sending metadata over a separate channel (e.g., a serial channel) could introduce inefficiencies and delays given the possibility that these separate channels are associated with different signal characteristics. Producing metadata frame alignment with video data in spite of these signal characteristic differences could involve the addition of a temporal calibration process, thereby increasing system complexity and reducing efficiency. While the disclosure herein makes reference to video frames, the techniques for “frame-alignment” described herein may also be used outside the context of video data, for example to enable efficient and rapid transmission of still-image data and temporally-associated metadata (e.g., camera acquisition metadata, camera uptime metadata, inertial measurement unit (IMU) metadata, endoscope metadata, etc.).

Further, various aspects of the present disclosure allow the system to diagnose errors that have occurred during the transmission and/or processing of the medical video data. For example, if the video frame is relayed by a series of devices, the data structure(s) received by the final device in the series of devices can include device identification data of each of the previous devices in the series of devices. Based on the device identification data, the system can determine the identities and order of the devices that were involved in generating, transmitting, and/or processing the video frame. Accordingly, the system can generate and provide a diagnostic report identifying the series of devices involved in generating, transmitting, and/or processing the video frame. If an error is identified in the video frame, the system can automatically determine where the error has originated in the series of devices.

Furthermore, some or all of the data generated using the techniques described herein may be transmitted to a remote device for further analytics. For example, the remote device can aggregate information about various errors with various video frames and, for each video frame, the series of devices that was involved in generating, processing, and transmitting the video frame. The remote device may further aggregate information about frame-specific metadata. The system can then identify associations between the data, such as an association between an error type and a device combination (e.g., the use of particular devices in the same series to transmit video data), an association between an error type and device configuration or usage (e.g., as indicated by frame-specific metadata), an association between an error type and a system configuration (e.g., the use of particular devices in a particular order), or any combination thereof. The identified associations can be used to improve the design, manufacturing, and deployment of devices to mitigate the errors. The identified associations can further be used to generate best practices for using and/or configurating devices and systems. For example, guidelines can be automatically provided to a system administrator as part of the instructions to properly set up, configure, and maintain devices and systems. The identified associations can further be used to diagnose errors that have occurred in the generation, transmission, and processing of video data.

Furthermore, various aspects of the present disclosure may generate and transmit a quality score for each video frame as part of the frame-specific metadata. The use of quality scores can facilitate visual documentation of surgical procedures and can be particularly advantageous for surgical procedures during which the camera may often be out-of-focus (e.g., due to relatively small or semi-rigid scopes). Embedding an image grab event and the quality score in the frame-specific metadata, which is sent synchronously with the corresponding video frame, can allow the system to select and output the best quality image without introducing additional points of failure in the hardware and without latency issues.

An exemplary method for transmitting and tracking the transmission of medical image data from a first device to a second device includes: receiving, at the first device, a video frame and frame-specific metadata; in response to receiving the video frame and the frame-specific metadata, generating, at the first device, device identification data of the first device; generating a set of one or more data structures in accordance with a predefined data specification, wherein the set of one or more data structures includes the frame-specific metadata and the device identification data; and transmitting, by the first device, the set of one or more data structures along with the video frame to the second device.

The method may be performed by a system including a series of devices communicatively coupled with each other and may further include relaying the video frame, by the series of devices, from an initial device of the series of devices to a final device of the series of devices, wherein the series of devices includes the first device and the second device, and wherein the second device follows the first device in the series of devices. The series of devices may include a camera configured to generate the video frame, a camera control unit, one or more encoders, one or more decoders, an image processing device, a display, or any combination thereof. The series of devices may include a third device following the second device, and the method may further include receiving, at the second device, the video frame and the set of one or more data structures; generating, at the second device, device identification data of the second device; updating, at the second device, the set of one or more data structures based on the device identification data of the second device; and transmitting, by the second device, the set of one or more data structures along with the video frame to the third device. Updating the set of one or more data structures may include generating a new data structure including the device identification data of the second device; and adding the new data structure to the set of one or more data structures. Updating the set of one or more data structures may include reading the set of one or more data structures; and adding the device identification data of the second device to a field of the set of one or more data structures.

The method may further include identifying an error in the video frame; and determining where the error originated based on the set of one or more data structures. The method may further include analyzing the video frame using a machine-learning model based on the set of one or more data structures. The set of one or more data structures may include one or more InfoFrame data structures defined by the predefined data specification. The set of one or more data structures may be transmitted during a blanking period during transmission of the video frame. The video frame may be acquired by a camera and the frame-specific metadata may include one or more parameters of the camera. The one or more parameters of the camera may include a gain parameter, an exposure parameter, an uptime parameter, a brightness parameter, a zoom parameter, an imaging mode parameter, a light pulse duration parameter, quaternion data, orientation data, a pitch angle, a roll angle, a raw angle, camera motion data, or any combination thereof.

The frame-specific metadata may include data related to one or more user inputs. The frame-specific metadata may include one or more checksum values associated with the video frame. At least one checksum value of the one or more checksum values may be specific to a color component of the video frame. The frame-specific metadata may include data related to an endoscope. The frame-specific metadata may include data indicative of a quality of the video frame. The quality of the video frame may be based on blurriness of the video frame, one or more artifacts in the video frame, brightness of the video frame, contrast of the video frame, or a weighted combination thereof.

An exemplary system for transmitting and tracking the transmission of medical image data from a first device to a second device includes: one or more processors, a memory, and one or more programs, wherein the one or more programs are stored in the memory and configured to be executed by the one or more processors, the one or more programs including instructions for: receiving, at the first device, a video frame and frame-specific metadata; in response to receiving the video frame and the frame-specific metadata, generating, at the first device, device identification data of the first device; generating a set of one or more data structures in accordance with a predefined data specification, wherein the set of one or more data structures includes the frame-specific metadata and the device identification data; and transmitting, by the first device, the set of one or more data structures along with the video frame to the second device.

The system may include a series of devices communicatively coupled with each other, and the one or more programs may further include instructions for relaying the video frame, by the series of devices, from an initial device of the series of devices to a final device of the series of devices, wherein the series of devices includes the first device and the second device, and wherein the second device follows the first device in the series of devices. The series of devices may include a camera configured to generate the video frame, a camera control unit, one or more encoders, one or more decoders, an image processing device, a display, or any combination thereof. The series of devices may include a third device following the second device, and the one or more programs further include instructions for receiving, at the second device, the video frame and the set of one or more data structures; generating, at the second device, device identification data of the second device; updating, at the second device, the set of one or more data structures based on the device identification data of the second device; and transmitting, by the second device, the set of one or more data structures along with the video frame to the third device. Updating the set of one or more data structures may include generating a new data structure including the device identification data of the second device; and adding the new data structure to the set of one or more data structures. Updating the set of one or more data structures may include reading the set of one or more data structures; and adding the device identification data of the second device to a field of the set of one or more data structures.

The one or more programs may further include instructions for identifying an error in the video frame; and determining where the error originated based on the set of one or more data structures. The one or more programs may further include instructions for analyzing the video frame using a machine-learning model based on the set of one or more data structures. The set of one or more data structures may include one or more InfoFrame data structures defined by the predefined data specification. The set of one or more data structures may be transmitted during a blanking period during transmission of the video frame. The video frame may be acquired by a camera and the frame-specific metadata may include one or more parameters of the camera. The one or more parameters of the camera may include a gain parameter, an exposure parameter, an uptime parameter, a brightness parameter, a zoom parameter, an imaging mode parameter, a light pulse duration parameter, quaternion data, orientation data, a pitch angle, a roll angle, a raw angle, camera motion data, or any combination thereof.

The frame-specific metadata may include data related to one or more user inputs. The frame-specific metadata may include one or more checksum values associated with the video frame. At least one checksum value of the one or more checksum values may be specific to a color component of the video frame. The frame-specific metadata may include data related to an endoscope. The frame-specific metadata may include data indicative of a quality of the video frame. The quality of the video frame may be based on blurriness of the video frame, one or more artifacts in the video frame, brightness of the video frame, contrast of the video frame, or a weighted combination thereof.

An exemplary non-transitory computer-readable storage medium stores one or more programs for transmitting and tracking the transmission of medical image data from a first device to a second device, the one or more programs including instructions, which when executed by one or more processors of an electronic device having a display, cause the electronic device to: receive, at the first device, a video frame and frame-specific metadata; in response to receiving the video frame and the frame-specific metadata, generate, at the first device, device identification data of the first device; generate a set of one or more data structures in accordance with a predefined data specification, wherein the set of one or more data structures includes the frame-specific metadata and the device identification data; and transmit, by the first device, the set of one or more data structures along with the video frame to the second device. The computer-readable storage medium may store instructions for performing any of the methods described above.

It will be appreciated that any one or more of the above aspects, features and options can be combined. It will be appreciated that any one of the options described in view of system apply equally to the imaging device, imaging controller or method, and vice versa.

### BRIEF DESCRIPTION OF THE FIGURES

The invention will now be described, by way of example only, with reference to the accompanying drawings, in which:

FIG. 1A shows an example of an endoscopic camera system, in accordance with some examples.

FIG. 1B shows an example of an open-field camera system, in accordance with some examples.

FIG. 2 illustrates an exemplary system comprising a series of electronic devices for generating, transmitting, and processing medical video data, in accordance with some examples.

FIG. 3 illustrates an exemplary process for transmitting and tracking the transmission of medical image data from a first device to a second device, in accordance with some examples.

FIG. 4 illustrates two exemplary devices of a series of devices for transmitting and tracking the transmission of medical image data, in accordance with some examples.

FIG. 5 illustrates an exemplary VSIF data structure, in accordance with some examples.

FIG. 6 illustrates exemplary blanking periods during transmission of a video frame, in accordance with some examples.

FIG. 7 illustrates exemplary contents of a data structure, in accordance with some examples.

FIG. 8 illustrates an exemplary process performed by a device for selecting a video frame based on the quality score, in accordance with some examples.

FIG. 9 illustrates an exemplary computer system in accordance with some examples.

### DETAILED DESCRIPTION

Reference will now be made in detail to implementations and examples of various aspects and variations of systems and methods described herein. Although several exemplary variations of the systems and methods are described herein, other variations of the systems and methods may include aspects of the systems and methods described herein combined in any suitable manner having combinations of all or some of the aspects described.

Described herein are devices, systems, and methods for generating and synchronously transmitting frame-specific metadata with medical image data (e.g., intraoperative video frames). An exemplary electronic device can obtain frame-specific metadata and device identification data of the electronic device, and generate one or more data structures (e.g., InfoFrames) in accordance with a predefined data specification. The electronic device can then transmit the one or more data structures along with a video frame to another electronic device.

Various aspects of the present disclosure provide several technical advantages. First, by generating data structures including frame-specific metadata and transmitting the data structures along with video frames, the systems described herein may ensure that video data and associated metadata are received, via transmission of the generated data structures, in a frame-aligned manner without the use of additional hardware. This assurance of temporal alignment of video frames and frame-specific metadata may be important for real-time surgical image processing techniques such as sensor alignment and image stabilization, as well as for post-processing procedures such as the conversion of video data and metadata to a different storage format, machine learning applications, and/or the quantification and normalization of values within a raw fluorescent image frame. Without the ability to send frame-specific metadata with video data on the same communication protocol, alternatives such as sending metadata over a separate channel (e.g., a serial channel) could introduce inefficiencies and delays given the possibility that these separate channels are associated with different signal characteristics. Producing metadata frame alignment with video data in spite of these signal characteristic differences could involve the addition of a temporal calibration process, thereby increasing system complexity and reducing efficiency. While the disclosure herein makes reference to video frames, the techniques for “frame-alignment” described herein may also be used outside the context of video data, for example to enable efficient and rapid transmission of still-image data and temporally-associated metadata.

Further, various aspects of the present disclosure allow the system to diagnose errors that have occurred during the transmission and/or processing of the medical video data. For example, if the video frame is relayed by a series of devices, the data structure(s) received by the final device in the series of devices can include device identification data of each of the previous devices in the series of devices. Based on the device identification data, the system can determine the identities and order of the devices that were involved in generating, transmitting, and/or processing the video frame. Accordingly, the system can generate and provide a diagnostic report identifying the series of devices involved in generating, transmitting, and/or processing the video frame. If an error is identified in the video frame, the system can automatically determine where the error has originated in the series of devices.

Furthermore, some or all of the data generated using the techniques described herein may be transmitted to a remote device for further analytics. For example, the remote device can aggregate information about various errors with various video frames and, for each video frame, the series of devices that was involved in generating, processing, and transmitting the video frame. The remote device may further aggregate information about frame-specific metadata. The system can then identify associations between the data, such as an association between an error type and a device combination (e.g., the use of particular devices in the same series to transmit video data), an association between an error type and device configuration or usage (e.g., as indicated by frame-specific metadata), an association between an error type and a system configuration (e.g., the use of particular devices in a particular order), or any combination thereof. The identified associations can be used to improve the design, manufacturing, and deployment of devices to mitigate the errors. The identified associations can further be used to generate best practices for using and/or configurating devices and systems. For example, guidelines can be automatically provided to a system administrator as part of the instructions to properly set up, configure, and maintain devices and systems. The identified associations can further be used to diagnose errors that have occurred in the generation, transmission, and processing of video data.

Furthermore, various aspects of the present disclosure may generate and transmit a quality score for each video frame as part of the frame-specific metadata. The use of quality scores can facilitate visual documentation of surgical procedures and can be particularly advantageous for surgical procedures during which the camera may often be out-of-focus (e.g., due to relatively small or semi-rigid scopes). Embedding an image grab event and the quality score in the frame-specific metadata, which is sent synchronously with the corresponding video frame, can allow the system to select and output the best quality image without introducing additional points of failure in the hardware and without latency issues.

In the following description of the various examples, reference is made to the accompanying drawings, in which are shown, by way of illustration, specific examples that can be practiced. It is to be understood that other aspects and examples can be practiced, and changes can be made without departing from the scope of the disclosure.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure in some examples also relates to a device for performing the operations herein. This device may be specially constructed for the required purposes, or it may comprise a general purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each connected to a computer system bus. Furthermore, the computers referred to in the specification may include a single processor or may be architectures employing multiple processor designs for increased computing capability.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein.

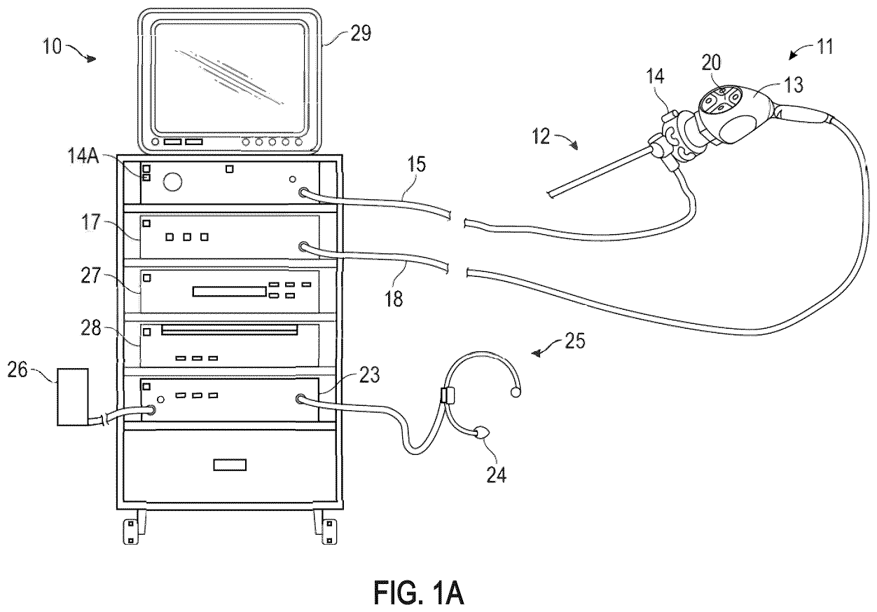

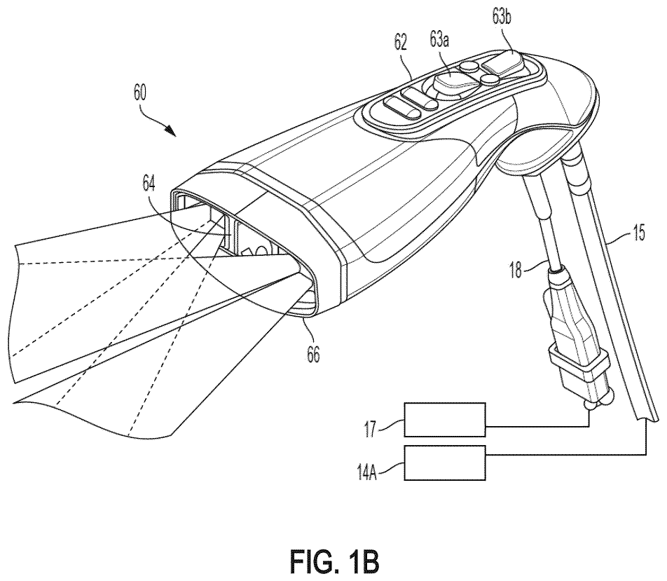

FIG. 1A shows an exemplary medical imaging system 10 that can utilize an, e.g. authenticable, data cable for connecting a medical imaging device to a medical imaging controller, according to the principles described herein. As used herein, medical imaging includes, but is not limited to, pre-operative, intra-operative, post-operative, and diagnostic imaging sessions and procedures. System 10 includes a scope assembly 11 which may be utilized in endoscopic procedures. The scope assembly 11 incorporates an endoscope or scope 12 which is coupled to an endoscopic camera head 13 by a coupler 14 located at the distal end of the camera head 13. Light is provided to the scope by a light source 14A via a light guide 15, such as a fiber optic cable. The camera head 13 is connected to a camera control unit (CCU) 17 by an electrical cable 18. Operation of the camera 13 is controlled, in part, by the CCU 17. The cable 18 conveys or transmits still and/or video image data from the camera head 13 to the CCU 17 and conveys various control signals bi-directionally between the camera head 13 and the CCU 17. In one example, the image data output by the camera head 13 is digital. The cable 18 may include a memory device for storing authentication data for authenticating the cable 18, as discussed further below.

A control or switch arrangement 20 may be provided on the camera head 13 and allows a user (e.g., surgeons, medical staff, and the like) to manually control various functions of the system 10. These and other functions may also be controlled by voice commands using a voice-control unit 23, which is connected to the CCU 17. Optionally, voice commands are input into a microphone 24 mounted on a headset 25 worn by the user and wiredly, or wirelessly, coupled to the voice-control unit 23. A hand-held control device 26, such as a tablet with a touch screen user interface or a PDA, may be connected to the voice control unit 23 as a further control interface. In the illustrated example, a recorder 27 and a printer 28 are also connected to the CCU 17. Additional devices, such as an image capture and archiving device, may be included in the system 10 and connected to the CCU 17. Video image data acquired by the camera head 13 and processed by the CCU 17 is converted to images, which can be displayed on a monitor 29, recorded by recorder 27, and/or used to generate static images, hard copies of which can be produced by printer 28.

FIG. 1B illustrates an open-field imaging device 60, which is another example of a type of imaging device that can be connected to an imaging controller via an, e.g. authenticable, cable, as discussed herein. Open-field imaging device 60 can be used as part of an imaging system, such as system 10 of FIG. 1A, for various purposes, including for visualizing blood flow in vessels and related tissue perfusion during plastic, microsurgical, reconstructive, and gastrointestinal procedures. As may be seen in FIG. 1B, the open-field imaging device 60 includes a control surface 62, a window frame 64 and a nosepiece 66. The open-field imaging device 60 is in this example connectable to the light source 14A via a light guide cable 15, through which the light is provided to the imaging field via ports in the window frame 64. The open-field imaging device 60 is connectable to the CCU 17 via an, e.g. authenticable, data cable 18, according to the principles described herein, which can transmit power, imaging data, and any other types of data.

The control surface 62 here includes focus buttons 63a (decreasing the working distance) and 63b (increasing the working distance) that control, e.g., outlet angles of the light beams for controlling a working distance at which the light beams substantially overlap for illuminating a target area. Other buttons on the control surface 62 may be programmable and may be used for various other functions, e.g., excitation laser power on/off, display mode selection, white light imaging white balance, saving a screenshot, and so forth. In some examples, the control surface functions can be communicated to the CCU 17 via non-imaging data communication lines in the cable 18, as discussed further below.

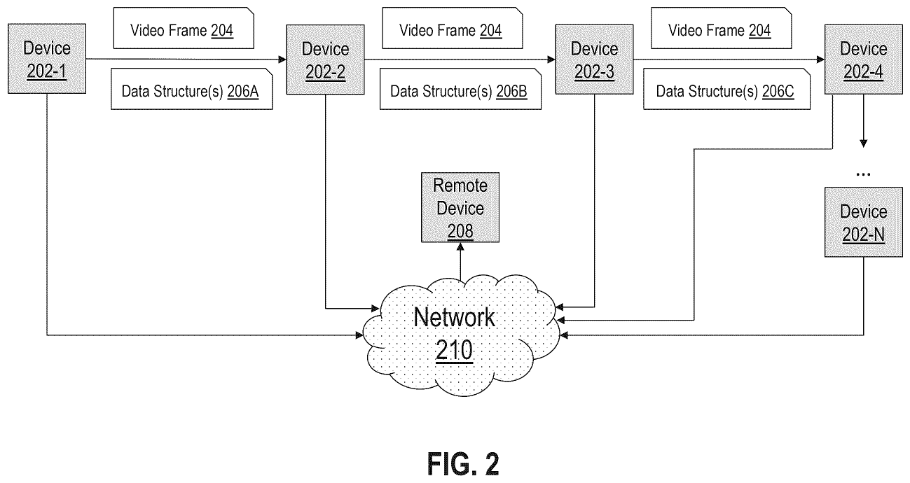

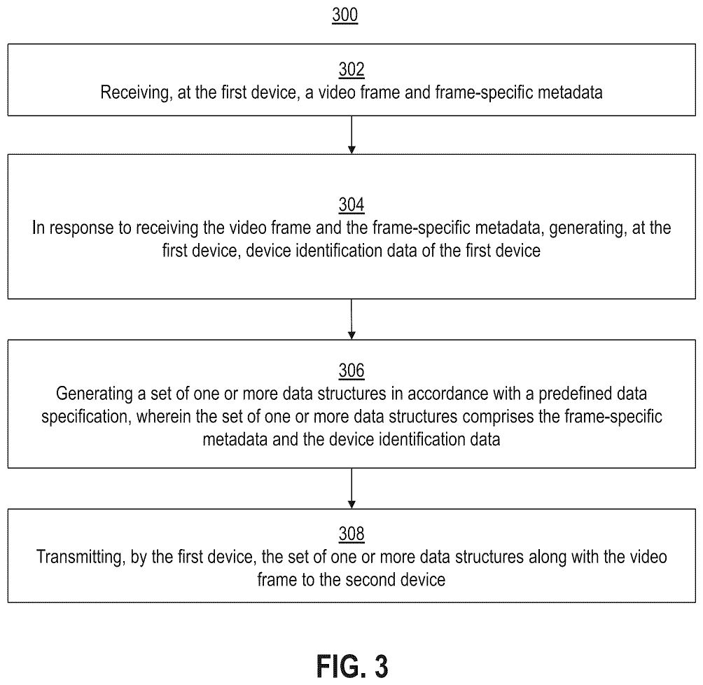

FIG. 2 illustrates an exemplary system 200 comprising a series of electronic devices for generating, transmitting, and processing medical video data, in accordance with some examples. The series of devices comprises an initial device 202-1 and a final device 202-N, as well as any number of devices between the initial device 202-1 and the final device 202-N(e.g., device 202-2, device 202-3, 202-4, etc.). The series of devices 202-1 through 202-N are communicatively coupled with each other. As shown, the device 202-1 can be configured to transmit data to the following device in the series (i.e., the device 202-2), which in turn can be configured to transmit data to the following device in the series (i.e., the device 202-3). The series of devices 202-1 through 202-N can be configured to relay a video frame and frame-specific metadata from the initial device to the final device in the series of devices, as described in detail with reference to FIG. 3.

In some examples, the initial device 202-1 in the series of devices is an imaging device, which can comprise a camera or camera head configured to generate a medical video frame and a CCU. In some examples, the imaging device can capture various types of visual information before, during, and after surgical procedures to assist a user (e.g., surgeons, medical staff, administrators, and the like) in planning, navigating, and performing surgeries. Exemplary surgical imaging data can include endoscopic imaging data (e.g., for visualizing the inside of organs and body cavities), fluorescence imaging data (e.g., for visualizing blood flow and tissue perfusion), X-ray imaging data, computed tomography (CT) imaging data, magnetic resonance imaging (MRI) data, ultrasound imaging data, optical coherence tomography (OCT) imaging data, or any combination thereof. In some examples, the surgical imaging data comprises at least one of pixel data and voxel data.

In some examples, the series of devices can comprise one or more encoders configured to convert the video frame from one format to another format for transmission, storage, and/or processing. In some examples, the series of devices can comprise one or more corresponding decoders. Suitable encoders and/or decoders may include, but are not limited to, HDMI to SDI converters, SDVoE converters, HDMI to AV converters, HDMI to DVI converters, HDMI to IP converters, and/or any other suitable types of converters.

In some examples, the series of devices can comprise one or more image processing devices configured to analyze and process the video frame. For example, a video processing device may comprise one or more algorithms to enhance, analyze, and/or interpret the video frame to assist in surgical planning, navigation, and execution. Exemplary algorithms can include image segmentation algorithms, image registration algorithms, image enhancement algorithms, image reconstruction algorithms, image fusion algorithms, image analysis and quantification algorithms, machine learning algorithms (e.g., detection algorithms, diagnosis algorithms), or any combination thereof. As described herein, the video processing device can be configured to apply one or more data processing operations to the received video data and the received frame-aligned metadata. The one or more data processing operations may include, for example, real-time surgical image processing such as sensor alignment and/or image stabilization. The one or more data processing operations may include, for example, post-processing procedures such as the conversion of video data and metadata to a different storage format, machine learning applications, and/or the quantification and normalization of values within a raw fluorescent image frame.

In some examples, the series of devices can comprise a display device. For example, the display device may be the final device 202-N in the series of devices. The display device can be configured to receive the video frame (e.g., after the video frame has been processed by an image processing device) and display the processed video frame and/or other results of the processing.

In some examples, the system 200 can include a remote device 208. Remote device 208 may be a computing system configured to analyze information received from one or more of devices 202-1 through 202-N. Remote device 208 may be located in the same environment or facility as devices 202-1 through 202-N(e.g., in a control room or storage closet of the facility) or in a different environment or facility (e.g., at a facility belonging to a third-party or affiliate, or a cloud computing service provider). The devices 202-1 through 202-N may be configured to transmit information to remote device 208 over a network 210. The information transmitted to remote device 208 may include frame-specific metadata, video frames, information about various errors with various video frames and, for each video frame, the series of devices that was involved in generating, processing, and transmitting the video frame. Remote device 208 may be configured to identify associations within the received information (e.g., associations between error types and device configurations). The identified associations can be used to improve the design, manufacturing, and deployment of devices to mitigate the errors.

FIG. 3 illustrates an exemplary process 300 for transmitting and tracking the transmission of medical image data from a first device to a second device, in accordance with some examples. Process 300 is performed, for example, using an exemplary system comprising two or more electronic devices (e.g., system 200 in FIG. 2). The system can provide efficient transmission of video data and associated metadata, and optionally for frame-by-frame processing of said video data and/or metadata, in accordance with some aspects. While some descriptions provided herein are directed to video data, it should be appreciated that any audiovisual data can be generated, transmitted, and processed in accordance with the techniques described herein. As used herein, “audiovisual data” may include: image and/or video data only, audio data only, and/or any combination thereof. As used herein, image and/or video data may include data representing electromagnetic radiation of any wavelength regardless of whether it is visible to the human eye.

In some examples, process 300 is performed using a client-server system, and the blocks of process 300 are divided up in any manner between the server and one or more client devices. In other examples, process 300 is performed using only a client device or only multiple client devices. In process 300, some blocks are, optionally, combined, the order of some blocks is, optionally, changed, and some blocks are, optionally, omitted. In some examples, additional steps may be performed in combination with the process 300. Accordingly, the operations as illustrated (and described in greater detail below) are exemplary by nature and, as such, should not be viewed as limiting.

In some examples, the process 300 is performed by a first device in a series of electronic devices communicatively coupled with each other, such as the system 200 in FIG. 2. The first device may be any device other than the final device in the series of devices, such as the initial device in the series of devices (e.g., device 202-1 in FIG. 2) or any device between the initial device and the final device in the series of devices (e.g., device 202-2, device 202-3, device 202-4, etc., in FIG. 2). How process 300 may be performed by various devices in the series of devices is provided in detail below.

### Process 300 Performed by the Initial Device in a Series of Devices

With reference to FIG. 3, at block 302, the first device receives a video frame and frame-specific metadata. If the first device is the initial device in the series of devices, the first device may be an imaging device that generates the video frame and metadata associated with the video frame in block 302. In the depicted example in FIG. 2, the first device performing block 302 may be the device 202-1, which may be an imaging device comprising a camera head configured to generate a video frame 204 and a CCU configured to generate metadata associated with the video frame 204.

At block 304, in response to receiving the video frame and the frame-specific metadata, the first device generates identification data of the first device. The identification data of the first device can include any information specifying the identity of the first device. In some examples, the identification data can include or specify a device type, a device name, a device ID, a serial number, a version number, a model number, firmware version, serial number, a network address (e.g., MAC address, IP address), a hardware ID, information related to the manufacturer of the device, information related to the functionalities of the device, configuration settings of the device, an uptime counter, a performance counter, resource information (e.g., CPU load, temperature), a Cyclic Redundancy Check (CRC) value, or any combination thereof. In the depicted example in FIG. 2, the first device performing block 304 may be the device 202-1, which may be an imaging device configured to generate identification data of the device 202-1 in block 304.

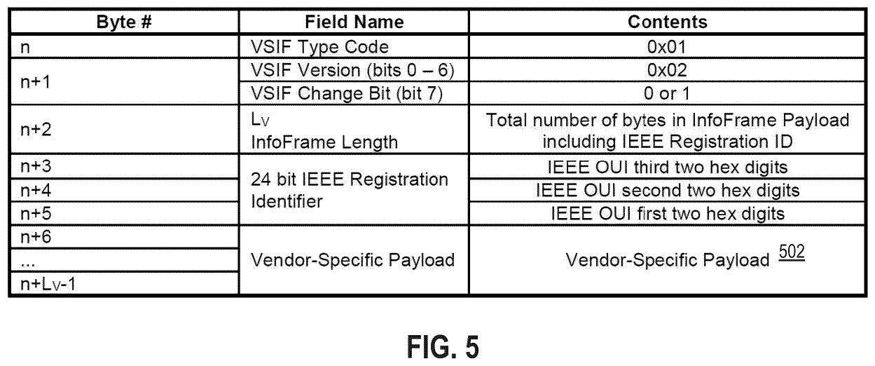

At block 306, the first device generates a set of one or more data structures in accordance with a predefined data specification. The set of one or more data structures can comprise the frame-specific metadata and the device identification data. The predefined data specification may be, for example, the HDMI specification. Data structures associated with the HDMI Specification can include, for example, the AVI InfoFrame, the Audio InfoFrame, and/or the MPEG Source InfoFrame. In some examples, the data structure is a Vendor Specific InfoFrame (VSIF). An InfoFrame refers to a type of metadata packet that accompanies video data to convey additional information about the video being transmitted. An exemplary structure of an InfoFrame is described herein, for example, with reference to FIG. 5.

In the depicted example in FIG. 2, the first device performing block 302 may be the device 202-1, which may be an imaging device comprising a camera head configured to generate a video frame 204 and a CCU configured to generate metadata associated with the video frame 204. At block 306, the device 202-1 can generate one or more data structures 206-1. The one or more data structures 206-1 may include one or more InfoFrame data structures encapsulating the metadata associated with the video frame 204 and the identification data of the device 202-1. In some examples, the device 202-1 may generate a single InfoFrame data structure, which includes both the metadata associated with the video frame 204 and the identification data of the device 202-1 in the payload of the InfoFrame data structure. In some examples, the device 202-1 may generate multiple InfoFrame data structures and the metadata associated with the video frame 204 and the identification data of the device 202-1 can be distributed across multiple payloads of the InfoFrame data structures.

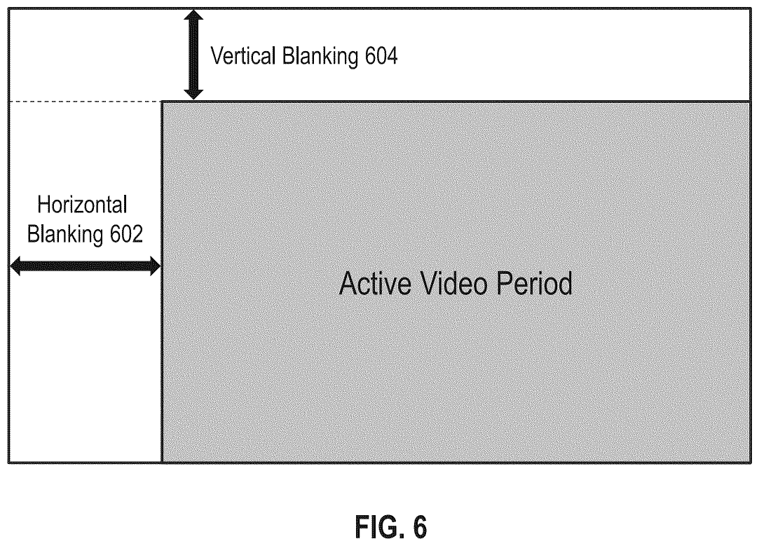

At block 308, the first device transmits the set of one or more data structures along with the video frame to a second device. The second device is the device that follows the first device in the series of devices. In the depicted example in FIG. 2, the first device performing block 302 may be the device 202-1, which can transmit the one or more data structures 206A along with the video frame 204 to the device 202-2. In some examples, the set of one or more data structures is transmitted during a blanking period during transmission of the video frame, as described in detail with reference to FIG. 6.

### Process 300 Performed by an Intermediate Device in a Series of Devices

As described above, the first device performing the process 300 may be any device other than the final device in the series of devices. Thus, the first device may be an intermediate device that is located between the initial device and the final device in the series of devices (e.g., device 202-2, device 202-3, device 202-4, etc., in FIG. 2). How process 300 may be performed by an intermediate device in the series of devices is provided in detail below.

With reference to FIG. 3, at block 302, the first device receives a video frame and frame-specific metadata. If the first device is an intermediate device in the series of devices, the first device may be configured to receive a video frame and frame-specific metadata from a previous device in the series of devices in block 302. In the depicted example in FIG. 2, the first device performing block 302 may be the device 202-2, which may be configured to receive the video frame 204 and data structures 206A from the previous device in the series of devices (i.e., the device 202-1).

At block 304, in response to receiving the video frame and the frame-specific metadata, the first device generates identification data of the first device. The identification data of the first device can include any information that specifies the identity of the first device. In some examples, the identification data can include or specify a device type, a device name, a device ID, a serial number, a version number, a model number, firmware version, serial number, a network address (e.g., MAC address, IP address), a hardware ID, information related to the manufacturer of the device, information related to the functionalities of the device, or any combination thereof. In the depicted example in FIG. 2, the first device performing block 304 may be the device 202-2, which can be configured to generate identification data of the device 202-2.

At block 306, the first device generates a set of one or more data structures in accordance with a predefined data specification. The set of one or more data structures can comprise the frame-specific metadata and the device identification data. The predefined data specification may be, for example, the HDMI specification. Data structures associated with the HDMI Specification can include, for example, the AVI InfoFrame, the Audio InfoFrame, and/or the MPEG Source InfoFrame. In some examples, the data structure is a Vendor Specific InfoFrame (VSIF). An InfoFrame refers to a type of metadata packet that accompanies video data to convey additional information about the video being transmitted. An exemplary structure of an InfoFrame is described herein, for example, with reference to FIG. 5.

In the depicted example in FIG. 2, the first device performing block 302 may be an intermediate device such as device 202-2. At block 306, the device 202-2 can generate one or more data structures 206B. The one or more data structures 206B may include one or more InfoFrame data structures encapsulating the metadata associated with the video frame 204 and the identification data of the device 202-2. In some examples, the device 202-2 can generate the data structure(s) 206B by adding new data (e.g., device identification data of device 202-2, any frame-specific metadata generated by the device 202-2) to data structure(s) 206A, which has been received from the device 202-1. In other words, the device 202-2 may not generate any new data structures, but instead update the payload of the existing data structure(s) 206A by adding the new data to the payload of the existing data structure(s) 206A. For example, device 202-2 may read the existing data structure(s) 206A and add device identification data of device 202-2 and/or frame-specific metadata generated by device 202-2 to a field of the existing data structure(s) 206A. Accordingly, the data structure(s) 206B may include the same number of InfoFrames as the data structure(s) 206A. In some examples, device 202-2 may not generate any new data structures or update the payload of existing data structures. Instead, device 202-2 may relay the existing data structure(s) 206A, without modification or update, to one or more subsequent devices in the series of devices (e.g., device 202-3).

Alternatively, the device 202-2 can generate one or more new data structures that encapsulate new metadata (e.g., device identification data of device 202-2, any frame-specific metadata generated by the device 202-2). Accordingly, the resulting data structure(s) 206B would include both the one or more new data structures and the existing data structure(s) 206A received from the device 202-1.

At block 308, the first device transmits the set of one or more data structures along with the video frame to a second device. The second device is the device that follows the first device in the series of devices. In the depicted example in FIG. 2, the first device performing block 302 may be the device 202-2, which can transmit the one or more data structures 206B along with the video frame 204 to the device 202-3. In some examples, the set of one or more data structures is transmitted during a blanking period during transmission of the video frame, as described in detail with reference to FIG. 6.

The process 300 can be performed by each intermediate device in the series of devices. In the depicted example, the process 300 can be performed by the device 202-2 to relay the video frame 204 and the frame-specific metadata (encapsulated in data structure(s) 206B) to the next device 202-3. Further, the process 300 can be performed by the device 202-3 to relay the video frame 204 and the frame-specific metadata (encapsulated in data structure(s) 206C) to the next device 202-4. Furthermore, the process 300 can be performed by the second-to-last device (not depicted) in the series to relay the video frame 204 and the frame-specific metadata to the final device 202-N.

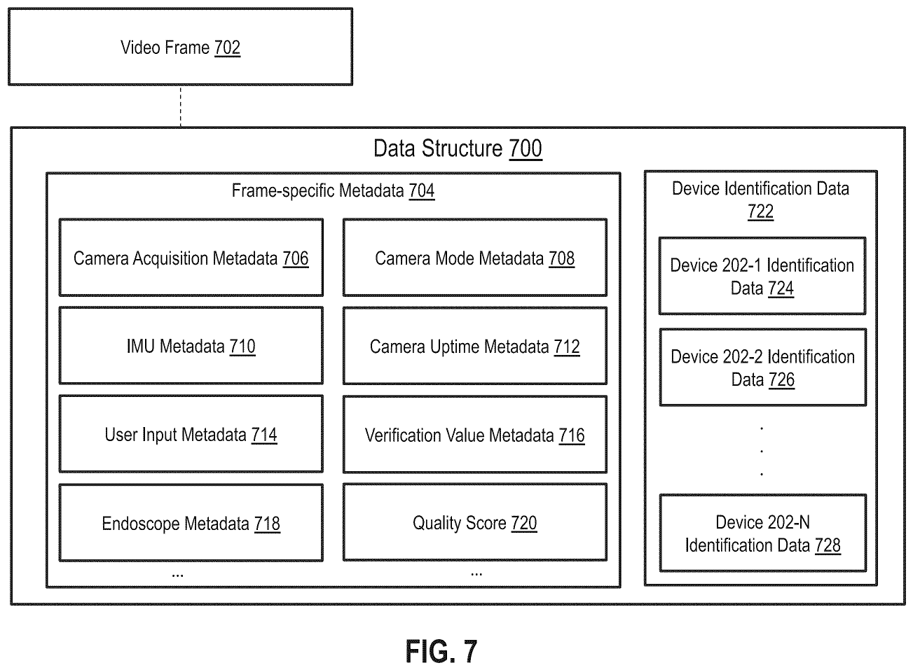

In some examples, the frame-specific metadata can be used to analyze the corresponding video frame (e.g., using a machine-learning model). Exemplary frame-specific metadata and the use thereof are provided herein with reference to, for example, FIG. 7. By generating data structures including frame-specific metadata that are transmitted along with video frames, the systems described herein may ensure that video data and associated metadata are received, via transmission of the generated data structures, in a frame-aligned manner. This assurance of temporal alignment of video frames and frame-specific metadata may be important for real-time surgical image processing techniques such as sensor alignment and image stabilization, as well as for post-processing procedures such as the conversion of video data and metadata to a different storage format, machine learning applications, and/or the quantification and normalization of values within a fluorescent image frame. Without the ability to send frame-specific metadata with video data on the same communication protocol, alternatives such as sending metadata over a separate channel (e.g., transmitting metadata asynchronously, such as via a serial channel) could introduce inefficiencies (e.g., increased latency in receiving metadata) given the possibility these separate channels are associated with different signal characteristics. Producing metadata frame alignment with video data in spite of these signal characteristic differences could involve the addition of a temporal calibration process, thereby increasing system complexity and reducing efficiency. While the disclosure herein makes reference to video frames, the techniques for “frame-alignment” described herein may also be used outside the context of video data, for example to enable efficient and rapid transmission of still-image data and temporally-associated metadata (e.g., camera acquisition metadata, camera uptime metadata, inertial measurement unit (IMU) metadata, endoscope metadata, etc.).

In some examples, the process 300 can allow the system to diagnose errors that have occurred during the transmission and/or processing of the medical video data. For example, the data structure(s) received by the final device can include device identification data of each of the previous devices in the series of devices (e.g., devices 202-1, 202-2, 202-3, 202-4, etc.). Based on the device identification data, the system can determine the identities and order of the devices that were involved in generating, transmitting, and/or processing the video frame. Accordingly, the system can generate and provide a diagnostic report identifying the series of devices involved in generating, transmitting, and/or processing the video frame.

If an error is identified in the video frame, the system can determine where the error has originated in the series of devices. For example, if a type of error in video data is known to be associated with a type of device, the system can then determine where the error may have originated from by identifying device(s) in the series of devices matching the device type (e.g., flickering video may be associated with a camera head, an encoder, a decoder, a display, etc.). As another example, the device identification data can be used together with the frame-specific metadata to diagnose an error. For example, the frame-specific metadata includes one or more checksum values associated with a video frame, which can indicate which device in the series of devices has caused the error. The device identification data can then be used to obtain further information about the error-originating device. The calculation and use of checksum values are described in detail with reference to FIG. 7.

In some examples, some or all of the data generated by the process 300, including the video frames, may be transmitted to a remote device (e.g., remote device 208) over a network (e.g., network 210) for further analytics. For example, the remote device can aggregate information about various errors with various video frames and, for each video frame, the series of devices that was involved in generating, processing, and transmitting the video frame. The remote device may further aggregate information about frame-specific metadata. The system can then identify associations between the data, such as an association between an error type and a device combination (e.g., the use of particular devices in the same series of devices), an association between an error type and device configuration or usage (e.g., as indicated by frame-specific metadata), an association between an error type and a system configuration (e.g., the use of particular devices in a particular order), or any combination thereof. The associations may be identified using one or more statistical models and/or machine-learning models, such as regression models, decision trees, random forests, support vector machines, K-nears neighbors, cluster analysis, principal component analysis (PCA), neural networks, etc.

In some examples, the identified associations can be used to improve the design, manufacturing, and deployment of devices to mitigate the errors. The identified associations can further be used to generate best practices for using and/or configurating devices and systems. For example, guidelines can be automatically provided to a system administrator as part of the instructions to properly set up, configure, and maintain devices and systems to avoid device or system configurations associated with known errors. The identified associations can further be used to diagnose errors that have occurred in the generation, transmission, and processing of video data.

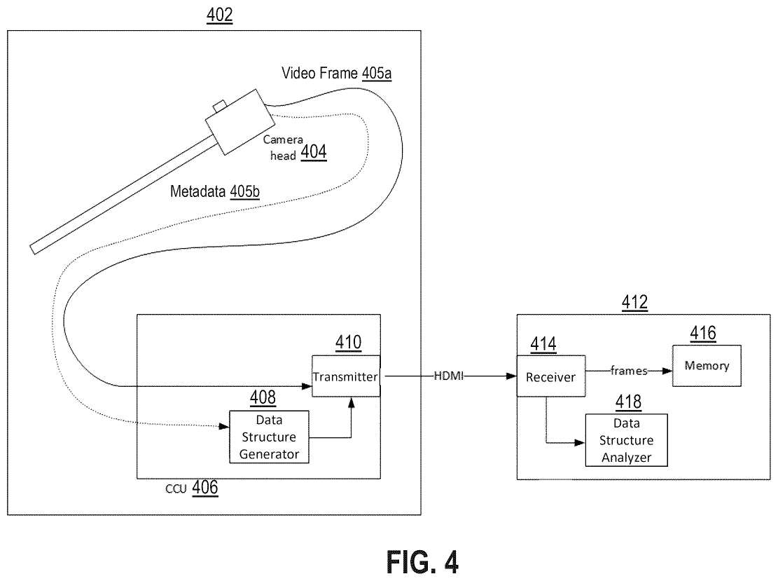

FIG. 4 illustrates two exemplary devices of a series of devices for transmitting and tracking the transmission of medical image data, in accordance with some examples. With reference to FIG. 4, the series of devices includes an imaging device 402 as the initial device. The imaging device 402 can comprise a camera head 404 and a CCU 406. The camera head 404 may include any one or more devices enabling the capture of medical or surgical audio and/or video, such as an audio and/or video capture device, a visible-light camera, a CCD or CMOS array, a photodiode array, a video-capture endoscope, an X-ray detector, an IR light detector, a UV light detector, and/or a microphone. At least a portion of imaging device 402 (e.g., an endoscope) may be pre-inserted into a body lumen. The methods of transmission of imaging metadata exclude the step of inserting at least a portion of an imaging device in a body lumen.

The camera head 404 can generate a video frame 405a and the corresponding frame-specific metadata 405b, which are provided to the CCU 406. Specifically, the video frame 405a is provided to a transmitter 410 of the CCU. In some examples, the transmitter 410 is an HDMI transmitter configured to send the video frame 405a as HDMI signals to another HDMI-enabled device. In some examples, the transmitter 410 is an SDI transmitter, a DVI transmitter, a VGA transmitter, an RCA transmitter, or any other suitable type of transmitter. Further, the metadata 405b is provided to a data structure generator 408 of the CCU. In some examples, device identification data of the imaging device 402 is also provided to the data structure generator 408. In some examples, the data structure generator 408 generates InfoFrame data structures. In some examples, the data structure generator 408 is a VSIF generator, which can generate one or more VSIF data structures encapsulating the frame-specific metadata 405b and the device identification data of the imaging device 402. The resulting one or more data structures are provided to the transmitter 410 for transmission along with the video frame 405a.

The next device in the series of devices is an image processing device 412. The image processing device 412 comprises a receiver 414. In some examples, the receiver 414 is an HDMI receiver configured to receive the video frame 405a and the one or more data structures as HDMI signals. In some examples, the receiver 414 is an SDI receiver, a DVI receiver, a VGA receiver, an RCA receiver, or any other suitable type of receiver. At the image processing device 412, the received video frame 405a can be provided to the memory 416 for storage. Further, the received data structures can be provided to a data structure analyzer 418 for decapsulation and analysis. As described herein, the received data structures can comprise device identification data of the imaging device 402 and frame-specific metadata 405b, which can be used to analyze the video frame 405a and/or diagnose errors associated with the video frame 405a.

FIG. 5 illustrates an exemplary VSIF data structure, in accordance with some examples. As shown, the VSIF data structure includes a vendor-specific payload field 502, which can be used to store and transmit device identification data or a portion thereof and/or frame-specific metadata or a portion thereof.

FIG. 6 illustrates exemplary blanking periods during transmission of a video frame, in accordance with some examples. Blanking periods refer to specific intervals of time within the data transmission (e.g., HDMI signal transmission) time period. With reference to FIG. 6, a horizontal blanking period 602 can occur before the active video transmission period within each horizontal line of the video signal. Further, a vertical blanking period 604 can occur between two frames, for example, between the transmission of the last horizontal line of the previous video frame and the transmission of the first horizontal line of the current video frame. During the blanking periods, no video data is transmitted. Instead, the one or more data structures encapsulating frame-specific metadata and/or device identification data may be transmitted during the blanking periods. It should be appreciated that, because the one or more data structures can be transmitted along the video frame data per the same protocol (e.g., HDMI protocol), there is no need for additional hardware components such as additional cables or connectors. In other examples, metadata is optionally transmitted as part of the video frame or over an audio channel associated with a predefined data specification.

In some examples, the frame-specific metadata may or may not be transmitted along with the corresponding video frame. For example, in FIG. 2, the data structure(s) 206A transmitted along with the video frame 204 (e.g., in a blanking period during the transmission of the video frame 204) may include frame-specific metadata associated with the video frame 204; alternatively, the data structure(s) 206A transmitted along with the video frame 204 may include frame-specific metadata associated with another video frame in the video stream (such as a video frame that is before the video frame 204 in the video stream).

In some examples, frame-specific metadata may be transmitted synchronously with the corresponding video frame using packetized transport (e.g., over IP). The frame-specific metadata may be partitioned into data packets. Information about the corresponding video frame may be included in the header for each data packet. The device receiving the data packet (e.g., device 202-2, device 202-3, device 202-4, device 202-N, or remote device 208) may then identify the video frame corresponding to the metadata based on the header for the data packet. The device receiving the data packet may extract the metadata from the data packet and use the metadata with the corresponding frame accordingly.

In some examples, frame-specific metadata for one video frame may need to be broken into multiple portions and transmitted across multiple blanking periods during the transmission of multiple video frames. The multiple portions can then be received and pieced together (e.g., at the final device in the series of devices) and used in downstream processing of the corresponding video frame. For example, with reference to FIG. 4, frame-specific metadata for a given video frame may be generated by camera head 404 and received at data structure generator 408 of CCU 406. The frame-specific metadata may be partitioned into a plurality of metadata packets by data structure generator 408 if the size of the frame-specific metadata for the given video frame exceeds the space allocated in vendor-specific payload field 502 of FIG. 5. The metadata packets may then be transmitted by transmitter 410 asynchronously with respect to the corresponding video frames.

In some examples, frame-specific metadata may or may not be generated for all video frames in a video stream. For example, with reference to FIG. 4, the imaging device 402 may generate frame-specific metadata associated with a first video frame in a video stream and forego generating additional frame-specific metadata for subsequent video frames if the same frame-specific metadata still applies to the subsequent video frames. For example, the camera head 404 of imaging device 402 may generate frame-specific metadata indicating a camera parameter for a first video frame in a video stream and, as long as the camera parameter remains the same, forego generating new frame-specific metadata indicating the camera parameter for subsequent video frames. The downstream processing of the subsequent video frames (e.g., by data structure analyzer 418 of image processing device 412) can rely on the camera parameter associated with the first video frame. When the camera parameter changes for a subsequent video frame, the camera head 404 may then generate new frame-specific metadata indicating the changed camera parameter (e.g., the new camera parameter, the difference between the old camera parameter and the new camera parameter).

FIG. 7 illustrates exemplary contents of a data structure 700, such as frame-specific metadata 704 and device identification data 722, in accordance with some examples. As described herein, a data structure 700 may include frame-specific metadata 704 associated with a video frame 702. The video frame 702 itself may not be included in data structure 700. The frame-specific metadata 704 associated with the video frame 702 can be generated or modified at each device that is involved in transmitting the video frame 702. For example, in the depicted example in FIG. 2, the frame-specific metadata 704 may be generated at the device 202-1 (i.e., the initial device in the series of devices) and transmitted to the device 202-2 in the one or more data structures 206A. At the device 202-2, the frame-specific metadata 704 may be modified (e.g., new verification values may be added as described below) and transmitted to the device 202-3 in the one or more data structures 206B, etc. At the device 202-N(i.e., the final device in the series of devices), the frame-specific metadata 704 may be used for analysis and processing of the video frame 702. In some examples, the frame-specific metadata 704 may be used for analysis and processing of the video frame 702 at an intermediate device (e.g., device 202-3) using the information provided by the previous devices (e.g., devices 202-1 and 202-2). With reference to FIG. 7, the frame-specific metadata 704 can include: camera acquisition metadata 706, camera mode metadata 708, inertial measurement unit (IMU) metadata 710, camera uptime metadata 712, user input metadata 714, verification value metadata 716, endoscope metadata 718, a quality score 720, or any combination thereof.

The camera acquisition metadata 706, the camera mode metadata 708, the IMU metadata 710, and the camera uptime metadata 712 can include or be related to parameters of the camera used to capture the video frame 702. The camera acquisition metadata 706 can include parameters related to the acquisition of the video frame 702, such as gain (e.g., gain for the red channel, green channel, blue channel, infrared channel, or the like), exposure (e.g., exposure for the red channel, green channel, blue channel, infrared channel, or the like), light pulse duration of the camera (e.g., light pulse duration for RGB illumination source, fluorescence excitation illumination source, or the like), focus setting of the camera (e.g., motorized focus setting and/or liquid lens focus setting), aperture setting of the camera, temperature of the camera, or any combination thereof. In some examples, the camera acquisition metadata 706 can be used for analyzing the video frame 702, such as for object detection and quantification of fluorescence in the video frame 702.

The camera mode metadata 708 can include parameters related to the mode of the camera used to capture the video frame 702, such as imaging mode (e.g., automatic mode, manual mode, overlay mode, white-light mode, fluorescence mode, or the like), specialty (e.g., arthroscopic camera, laparoscopic camera, or the like), user-specified camera settings, brightness level, zoom level, HDR tone mode, focus settings, or any combination thereof.

The IMU metadata 710 can include parameters related to the position, orientation, and/or motion of the camera used to capture the video frame 702, such as quaternions, pitch angle, roll angle, yaw angles, data related to IMU sensors (gyroscope, accelerometer, magnetometer, or the like), or any combination thereof. In some examples, the IMU metadata 710 can be used for image stabilization, horizon-leveling, image stitching (e.g., for selecting an image associated with the least amount of motion), or any combination thereof.

The camera uptime metadata 712 can include information related to the amount of time the camera has been operational or available for use without experiencing downtime or interruptions. The uptime may be measured in terms of time (e.g., seconds, minutes, hours, days,) or frame counts. In some examples, the camera uptime metadata 712 can include information related to the maximum duration of the camera.

Further with reference to FIG. 7, the frame-specific metadata 704 can include user input metadata 714, which includes data related to one or more user inputs, such as an image grab event (e.g., a command from a user via a suitable user interface for capturing a still image), a button press (e.g., state of the buttons on the camera head), or any combination thereof. As described below, the image grab event can be used with the quality score to select a video frame from a plurality of video frames for output.

Further with reference to FIG. 7, the frame-specific metadata 704 can include verification value metadata 716, which includes one or more numeric values (e.g., a checksum value, a hash value, a Cyclic Redundancy Check (CRC) value, or the like) associated with the video frame for error-checking purposes. In some examples, after a device receives a video frame (e.g., at block 302 in FIG. 3), the device calculates one or more verification values (e.g., a checksum value, a hash value, and/or a Cyclic Redundancy Check (CRC) value) for the video frame. In some examples, the device may calculate one or more verification values for each color component of the video frame (e.g., red component, green component, blue component). In some examples, the device can calculate verification values for the same video frame twice—once before processing the video frame (e.g., upon receiving the video frame) and once after processing the video frame at the device (e.g., before transmitting the processed video frame to the next device).

The verification value metadata 716 can be used to diagnose an error with the video frame, for example, to identify which device in the series of devices that error has originated from. For example, if a device is not configured to make changes to a video frame, the verification value is then not expected to change before and after the device processes the video frame (e.g., when the device receives the video frame v. when the device transmits the video frame to the next device). Thus, a change in the verification values may indicate that an error has occurred on the device (e.g., the video frame has been inadvertently modified by the device). As another example, if the video frame is transmitted from a first device to a second device and the verification value calculated by the first device differs from the verification value calculated by the second device, the difference in the verification values may indicate that an error has occurred during the transmission (e.g., the data is corrupted or altered during the transmission) between the two devices. If the verification value is calculated specific to a color component, the system can determine on which transmission line or wire the error has occurred.

Further with reference to FIG. 7, the frame-specific metadata 704 can include endoscope metadata 718, which can include data related to an endoscope of the imaging system, such as the location of the endoscope (e.g., x, y coordinates of a reference point on the endoscope), radius of the endoscope, identification information of the endoscope (device ID, model number), or any combination thereof. In some examples, the endoscope metadata 718 can be used for scope edge detection in the video frame 702. In some examples, the endoscope metadata 718 can be used for optical calibration of the video frame 702. For example, the calculation of the transformation matrix during calibration can be based on parameters associated with a specific type of endoscope. As another example, based on the endoscope metadata 718, the system can detect when a new endoscope is in use and perform recalibration.

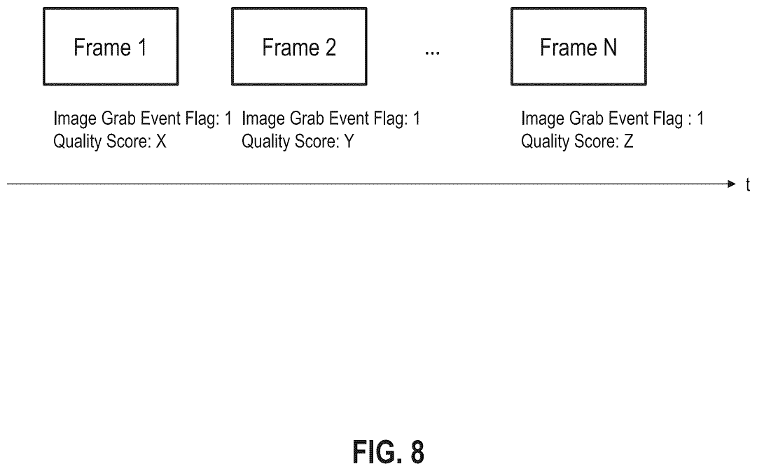

Further with reference to FIG. 7, the frame-specific metadata 704 can include a quality score 720 indicative of the quality of the image captured in the video frame 702. The quality score can be determined based on blurriness of the image captured in the video frame 702, one or more artifacts in the video frame 700, brightness of the video frame 702, contrast of the video frame 702, or a weighted combination thereof. If the system detects a user input to obtain a captured video frame from the video stream (e.g., based on user input metadata 714), the system (e.g., data structure analyzer 418 of image processing device 412) can select a video frame from a plurality of video frames based on the quality scores associated with the video frames, as described in detail with reference to FIG. 8.

The selection of a video frame based on the quality score can be performed by any image processing device, such as a device in the series of devices depicted in FIGS. 2 and 4. For example, with reference to FIG. 4, a user may activate a button on the camera head 404 to trigger a still image grab for documentation purposes during a surgical procedure. At the camera head 404, a video frame 405a is captured and the frame-specific metadata 405b can include the image grab event (e.g., as part of the user input metadata 714 in FIG. 7). The CCU 406 can determine the quality of the video frame 405a by calculating a quality score based on blurriness of the video frame 405a (e.g., using a Laplacian filter, Fast Fourier Transform), one or more artifacts in the video frame 405a, brightness of the video frame 405a, contrast of the video frame 405a, or a weighted combination thereof. The quality score can be included in the one or more data structures, which are transmitted along with the video frame 405a to a downstream image processing device 412. In some examples, the system may continue to include the image grab event in the frame-specific metadata for a number of video frames captured immediately after the video frame 405a (e.g., within a time period or a predefined number of video frames) such that a selection can be made based on quality scores downstream, as described with reference to FIG. 8.

FIG. 8 illustrates an exemplary process performed by a device (e.g., image processing device 412) for selecting a video frame based on quality scores, in accordance with some examples. With reference to FIG. 8, the device receives a video stream comprising a series of video frames (Frame 1, Frame 2, . . . Frame N) over time. Each video frame is associated with frame-specific metadata, which can indicate whether the video frame is to be included for selection for an image grab output (e.g., via an image grab event flag) and the quality score associated with the video frame. In the depicted example, the image grab flag is a binary value. As described above, after the user activates a button on the camera head to trigger a still image grab, the frame-specific metadata for multiple video frames (e.g., within a predefined time period, a predefined number of video frames) may indicate the image grab event (e.g., via the image grab flag) so that those video frames can be included for selection for an image grab output. In the depicted example, the device can select a video frame having the highest quality score out of the video frames 1-N with a set image grab flag.

The use of quality scores can facilitate visual documentation of surgical procedures and can be particularly advantageous for surgical procedures during which the camera may often be out-of-focus (e.g., due to relatively small or semi-rigid scopes). During these surgical procedures, a slight movement may result in a blurry image, making it difficult for users (e.g., surgeons, medical staff, administrators, and the like) to reliably obtain clear image frames from the video stream to document their work. For example, with reference to FIG. 4, in a conventional workflow, the user may activate a button on the camera head 404 to trigger a still image grab. The camera head 404 can send the image grab event (e.g., in the form of an electrical pulse signal) to the CCU 406 that, in turn, sends that image grab event to the image processing device 412. Upon receiving the image grab event, the image processing device 412 extracts one video frame from the video stream received on a video port. The conventional workflow is deficient for several reasons. First, because many video frames are blurry in the video stream, it is likely that the extracted video frame may be blurry. Further, the video stream (e.g., HDMI video stream) and the image grab event are asynchronous, thus requiring a latency verification test to make sure the actual image grab occurs within a reasonable time frame of the user pressing the capture button on the camera head 404. Further still, having separate physical ports for video stream and image grab events creates an additional potential point of failure, necessitating prolonged verification and validation time and resources, requiring more prime real estate at the back of both devices, and raising the cost of the overall platform. Embedding the image grab event in the frame-specific metadata, which is sent synchronously with the corresponding video frames, can address latency uncertainty. Further, the quality score can allow the best quality image to be selected for output.

Returning to FIG. 7, a data structure 700 may further include device identification data 722. Each device in a series of devices (e.g., device 202-1, device 202-2 . . . device 202-N) may generate device identification data 722 corresponding to the respective device. The device identification data 722 may include any information specifying the identity of the device(s) involved in generating, transmitting, and/or processing a video frame 702. For example, the device identification data may include device 202-1 identification data 724, device 202-2 identification data 726, and so on up to device 202-N identification data 728. The device identification data 722 corresponding to each device may include or specify a device type, a device name, a device ID, a serial number, a version number, a model number, firmware version, serial number, a network address (e.g., MAC address, IP address), a hardware ID, information related to the manufacturer of the device, information related to the functionalities of the device, configuration settings of the device, an uptime counter, a performance counter, resource information (e.g., CPU load, temperature), a Cyclic Redundancy Check (CRC) value, or any combination thereof. Optionally, the device identification data may further include identification data for remote device 208. Device identification data for remote device 208 may be useful, for example, when remote device 208 runs a machine learning algorithm and sends associated metadata through the series of devices 202-1 through 202-N.

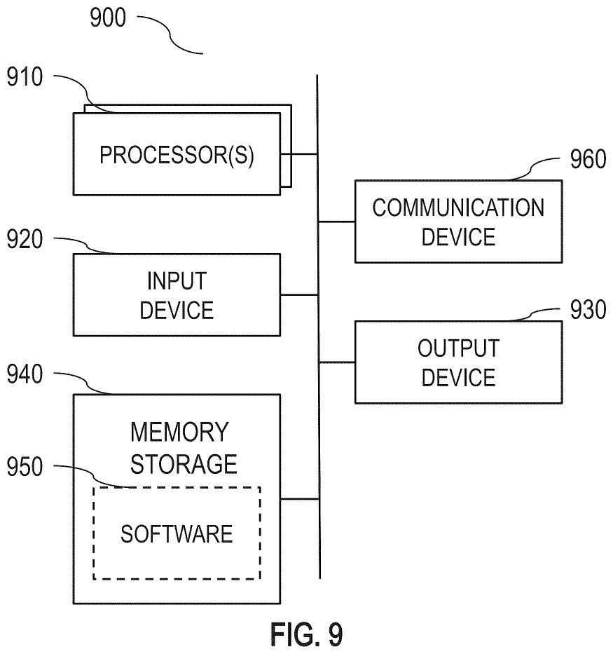

The operations described herein are optionally implemented by components depicted in FIG. 9. FIG. 9 illustrates an example of a computing device. Device 900 can be a host computer connected to a network. Device 900 can be a client computer or a server. As shown in FIG. 9, device 900 can be any suitable type of microprocessor-based device, such as a personal computer, workstation, server or handheld computing device (portable electronic device) such as a phone or tablet. The device can include, for example, one or more of processor 910, input device 920, output device 930, storage 940, and communication device 960. Input device 920 and output device 930 can generally correspond to those described above, and can either be connectable or integrated with the computer.

Input device 920 can be any suitable device that provides input, such as a touch screen, keyboard or keypad, mouse, or voice-recognition device. Output device 930 can be any suitable device that provides output, such as a touch screen, haptics device, or speaker.

Storage 940 can be any suitable device that provides storage, such as an electrical, magnetic or optical memory including a RAM, cache, hard drive, or removable storage disk. Communication device 960 can include any suitable device capable of transmitting and receiving signals over a network, such as a network interface chip or device. The components of the computer can be connected in any suitable manner, such as via a physical bus or wirelessly.

Software 950, which can be stored in storage 940 and executed by processor 910, can include, for example, the programming that embodies the functionality of the present disclosure (e.g., as embodied in the devices as described above).

Software 950 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 940, that can contain or store programming for use by or in connection with an instruction execution system, apparatus, or device.

Software 950 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 900 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 900 can implement any operating system suitable for operating on the network. Software 950 can be written in any suitable programming language, such as C, C++, Java or Python. Application software embodying the functionality of the present disclosure can be deployed in different configurations, such as in a client/server arrangement or through a Web browser as a Web-based application or Web service, for example.

The foregoing description, for the purpose of explanation, has been described with reference to specific examples. However, the illustrative discussions above are not intended to be exhaustive or to limit the invention to the precise forms disclosed. Many modifications and variations are possible in view of the above teachings. The examples were chosen and described in order to best explain the principles of the techniques and their practical applications. Others skilled in the art are thereby enabled to best utilize the techniques and various examples with various modifications as are suited to the particular use contemplated. For the purpose of clarity and a concise description, features are described herein as part of the same or separate examples, however, it will be appreciated that the scope of the invention may include examples having combinations of all or some of the features described.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A method for transmitting and tracking the transmission of medical image data from a first device to a second device, comprising:
receiving, at the first device, a video frame and frame-specific metadata;
in response to receiving the video frame and the frame-specific metadata, generating, at the first device, device identification data of the first device;
generating a set of one or more data structures in accordance with a predefined data specification, wherein the set of one or more data structures comprises the frame-specific metadata and the device identification data; and
transmitting, by the first device, the set of one or more data structures along with the video frame to the second device.

2. The method of claim 1, wherein the method is performed by a system comprising a series of devices communicatively coupled with each other, the method further comprising:
relaying the video frame, by the series of devices, from an initial device of the series of devices to a final device of the series of devices,
wherein the series of devices comprises the first device and the second device, and
wherein the second device follows the first device in the series of devices.

3. The method of claim 2, wherein the series of devices comprises:
a camera configured to generate the video frame,
a camera control unit,
one or more encoders,
one or more decoders,
an image processing device,
a display, or
any combination thereof.

4. The method of claim 2, wherein the series of devices comprises a third device following the second device, the method further comprising:
receiving, at the second device, the video frame and the set of one or more data structures;
generating, at the second device, device identification data of the second device;
updating, at the second device, the set of one or more data structures based on the device identification data of the second device; and
transmitting, by the second device, the set of one or more data structures along with the video frame to the third device.

5. The method of claim 4, wherein updating the set of one or more data structures comprises:
generating a new data structure comprising the device identification data of the second device; and
adding the new data structure to the set of one or more data structures.

6. The method of claim 4, wherein updating the set of one or more data structures comprises:
reading the set of one or more data structures; and
adding the device identification data of the second device to a field of the set of one or more data structures.

7. The method of claim 1, further comprising:
identifying an error in the video frame; and
determining where the error originated based on the set of one or more data structures.

8. The method of claim 1, further comprising: analyzing the video frame using a machine-learning model based on the set of one or more data structures.

9. The method of claim 1, wherein the set of one or more data structures comprises one or more InfoFrame data structures defined by the predefined data specification.

10. The method of claim 1, wherein the set of one or more data structures is transmitted during a blanking period during transmission of the video frame.

11. The method of claim 1, wherein the video frame is acquired by a camera and wherein the frame-specific metadata comprises: one or more parameters of the camera, wherein the one or more parameters of the camera comprise: a gain parameter, an exposure parameter, an uptime parameter, a brightness parameter, a zoom parameter, an imaging mode parameter, a light pulse duration parameter, quaternion data, orientation data, a pitch angle, a roll angle, a raw angle, camera motion data, or any combination thereof.

12. The method of claim 1, wherein the frame-specific metadata comprises data related to one or more user inputs.

13. The method of claim 1, wherein the frame-specific metadata comprises one or more checksum values associated with the video frame, wherein at least one checksum value of the one or more checksum values is specific to a color component of the video frame.

14. The method of claim 1, wherein the frame-specific metadata comprises data related to an endoscope.

15. The method of claim 1, wherein the frame-specific metadata comprises data indicative of a quality of the video frame, wherein the quality of the video frame is based on blurriness of the video frame, one or more artifacts in the video frame, brightness of the video frame, contrast of the video frame, or a weighted combination thereof.

16. A system for transmitting and tracking the transmission of medical image data from a first device to a second device, comprising:
one or more processors,
a memory, and
one or more programs, wherein the one or more programs are stored in the memory and configured to be executed by the one or more processors, the one or more programs including instructions for:
receiving, at the first device, a video frame and frame-specific metadata;
in response to receiving the video frame and the frame-specific metadata, generating, at the first device, device identification data of the first device;
generating a set of one or more data structures in accordance with a predefined data specification, wherein the set of one or more data structures comprises the frame-specific metadata and the device identification data; and
transmitting, by the first device, the set of one or more data structures along with the video frame to the second device.

17. The system of claim 16, comprising a series of devices communicatively coupled with each other, the one or more programs further including instructions for:
relaying the video frame, by the series of devices, from an initial device of the series of devices to a final device of the series of devices,
wherein the series of devices comprises the first device and the second device, and
wherein the second device follows the first device in the series of devices.

18. The system of claim 16, the one or more programs further including instructions for:
identifying an error in the video frame; and
determining where the error originated based on the set of one or more data structures.

19. The system of claim 16, wherein the set of one or more data structures is transmitted during a blanking period during transmission of the video frame.

20. A non-transitory computer-readable storage medium storing one or more programs for transmitting and tracking the transmission of medical image data from a first device to a second device, the one or more programs comprising instructions, which when executed by one or more processors of an electronic device having a display, cause the electronic device to:
receive, at the first device, a video frame and frame-specific metadata;
in response to receiving the video frame and the frame-specific metadata, generate, at the first device, device identification data of the first device;
generate a set of one or more data structures in accordance with a predefined data specification, wherein the set of one or more data structures comprises the frame-specific metadata and the device identification data; and
transmit, by the first device, the set of one or more data structures along with the video frame to the second device.
